(19) United States
(12) Patent Application Publication
(10) Pub. No.: US 2016/0109460 A1
(43) Pub. Date: Apr. 21, 2016

(54) Methods And Compositions For Determining Fortilin Levels

(71) Applicant: Ken Fujise, Galveston, TX (US)

(72) Inventor: Ken Fujise, Galveston, TX (US)

(73) Assignee: The Board of Regents of the University of Texas System, Austin, TX (US)

(21) Appl. No.: 14/883,606

(22) Filed: Oct. 14, 2015

Related U.S. Application Data
(60) Provisional application No. 62/064,887, filed on Oct. 16, 2014.

(51) Int. Cl. G01N 33/68

(52) U.S. Cl. CPC G01N 33/6893; G01N 2800/52; G01N 2333/4703

(57) ABSTRACT

Certain embodiments are directed to method of measuring fortilin in a serum sample as a biomarker of in vivo apoptosis, which can be utilized to noninvasively assess the status of in vivo apoptosis in a subject.

This application claims priority to U.S. Provisional Application 62/064,887 filed Oct. 16, 2014, which is incorporated herein by reference in its entirety.

### STATEMENT REGARDING FEDERALLY FUNDED RESEARCH

This invention was made with government support under UL1TR000071, 5R01CA127971, and R01HL117247 awarded by the National Institutes of Health, National Cancer Institute, and National Heart Lung and Blood Institute, respectively. The government has certain rights in the invention.

### BACKGROUND

Approximately 50 to 70 billion cells undergo apoptosis each day in the average normal adult (Reed, Journal of Clinical Oncology, 17 (1999) 2941-2953). Serum biomarkers of apoptosis—molecules that can be readily and objectively measured as indicators of normally and pathologically occurring apoptosis at tissue and organ levels—would allow clinicians to easily monitor the status of apoptosis associated with the diseases and conditions they treat—such as apoptosis-induced skeletal muscle atrophy resulting from cancer (cachexia), aging (sarcopenia) (Dupont-Versteegden, Exp Gerontol, 40 (2005) 473-481), starvation, denervation, disuse, and inflammation (Schwartz, Cell Death Differ, 15 (2008) 1163-1169). Cancer cells undergo apoptosis at a higher rate than do normal cells and massively apoptose in response to radiation therapy and chemotherapy (Ulukaya et al., Lung cancer, 56 (2007) 399-404). Serum biomarkers of apoptosis could thus allow clinicians to screen patients for certain cancers or to monitor the response of patients with cancer to anticancer chemotherapy or radiation therapy (Ward et al., Br J Cancer, 99 (2008) 841-46).

Thus far, three serum biomarkers of apoptosis have been reported in the literature including the fragmented cytokeratin-18 (fCK18, detectable by the M30 antibody), nucleosomally-cleaved genomic DNA (n-DNA), and cytochrome c (Cyt C) (Ward et al., Br J Cancer, 99 (2008) 841-46)—each with notable limitations to their utility. The utility of fCK18 is limited to apoptosis occurring in cells of epithelial origin (Caulin et al., J Cell Biol, 138 (1997) 1379-1394). The utility of circulating n-DNA is diminished because it can be rapidly degraded by serum DNases (Tamkovich et al., Ann N Y Acad Sci, 1075 (2006) 191-196). Cyt C is reportedly released from both apoptotic (Renz et al., Blood, 98 (2001) 1542-48) and necrotic cells (Jemmerson et al., Cell Death Differ, 9 (2002) 538-48), depending on the extent of cellular damage, thus limiting its specificity. Further, these candidate serum apoptosis biomarkers have not been extensively characterized or validated at clinical, whole animal, and cellular levels.

### SUMMARY

Using a newly developed fortilin ELISA system, the inventors show here that fortilin exists in the normal human and mouse circulation, and further demonstrate that fortilin serum levels are significantly elevated in patients with solid cancer and/or in response to anti-cancer chemotherapy or radiation therapy. The elevation of fortilin serum levels is more robust and sensitive than that of previously-reported serum biomarkers of apoptosis, e.g., fragmented cytokeratin-18, cytochrome c, and nucleosomal DNA. Serum fortilin levels reflect the degree and extent of apoptosis occurring in vivo. Thus, fortilin is a serum biomarker of in vivo apoptosis and can be utilized to noninvasively assess the status of in vivo apoptosis in a subject, e.g., a human.

Certain embodiments are directed to assays for detecting and/or measuring fortilin in a biological sample. Other embodiments are directed to detecting apoptosis or determining the level of apoptosis by measuring fortilin levels in a biological sample, with elevated levels of fortilin being indicative of increased apoptosis.

Certain embodiments are directed to methods of measuring fortilin in a blood sample comprising contacting a blood sample with an antibody that specifically binds fortilin forming an antibody/fortilin complex; contacting the antibody/fortilin complex with a detection reagent; and measuring the amount of detection reagent bound to the antibody/fortilin complex. In certain aspects the antibody that specifically binds fortilin is immobilized on a support, e.g., a microtiter plate. In other aspects the amount of detection reagent is determined by a chemical or enzymatic reaction.

Certain embodiments are directed to methods for determining the level of in vivo apoptosis in a subject comprising measuring fortilin levels in a blood sample from the subject and determining whether the fortilin levels exceed a predetermined threshold representing levels of fortilin in a subject not having a disease, condition, or undergoing a therapy, wherein elevated fortilin levels are indicative of increased apoptosis associated with the disease, condition, or therapy. In certain aspects the subject is undergoing cancer therapy, such as chemotherapy or radiation therapy. In a further aspect the subject is suspected of having apoptosis-induced muscle atrophy. In certain aspects the disease or condition is cachexia, skeletal muscle atrophy associated with aging (sarcopenia), skeletal muscle atrophy associated with starvation, skeletal muscle atrophy associated with denervation, skeletal muscle atrophy associated with disuse, and skeletal muscle atrophy associated with inflammation. In certain aspects subject is a human.

A “biological sample,” as used herein, generally refers to a sample of tissue or fluid from a human or animal including, but not limited to plasma, serum, spinal fluid, lymph fluid, the external sections of the skin, respiratory, intestinal and genitourinary tracts, tears, nasal secretions, saliva, blood cells, tumors, organs, tissue and sample of in vitro cell culture constituents. In certain aspects the biological sample is a blood sample or a fraction thereof.

“Subject,” as used herein, refers to humans or non-human animals. In certain aspects the subject is human.

The term “antibody,” as used herein generally refers to antibodies, digestion fragments, specified portions and variants thereof, including antibody mimetics or comprising portions of antibodies that mimic the structure and/or function of an antibody or specified fragment or portion thereof, including single chain antibodies and fragments thereof. The invention encompasses antibodies and antibody fragments capable of binding to a biological molecule (such as an antigen or receptor), such as fortilin or portions thereof.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Development and characterization of fortilin ELISA. Abbreviations: Ab, antibody; HRP, horseradish peroxidase; TMB, 3,3′,5,5′-tetramethylbenzidine. (A) The design of fortilin ELISA. (B) Detection limits of the fortilin ELISA. Each span of the error bar represents 3× standard deviation (SD). (C) Histogram of mouse serum fortilin levels. (D) Probability plot of mouse serum fortilin levels using the normal distribution fit with the 5th and 95th percentile values of 26.84 and 69.48 ng/mL. (E) Histogram of human serum fortilin levels. (F) Probability plot of human serum fortilin levels using the gamma distribution fit with 5th and 95th percentile values of 18.68 and 163.55 ng/mL.

FIG. 2. Serum fortilin levels are increased after anti-cancer therapy (radiation therapy, chemotherapy, or both). Abbreviations: *, statistically significant (P<0.05); PRE, pre-anti-cancer therapy; POST, post-anti-cancer therapy. (A) Change in serum fortilin levels [ng/mL] in 18 patients undergoing anti-cancer therapy. The details of the patients are in Table 1 below. (B) Change in serum fortilin levels when pre-treatment levels were normalized to one. Patient #10: a sufficient amount of post-treatment sample was available only for fortilin ELISA, thus included for serum fortilin assay but excluded from the other assays. Patient #5: excluded from the study as post-treatment sample volume was not sufficient for any assays.

FIG. 3. Infrequent increases in serum lactate dehydrogenase (LDH) levels suggest a lack of extensive necrotic tissue damage in the study patients. Abbreviations: LDH, lactate dehydrogenase; *, P<0.05; PRE, pre-anti-cancer therapy; POST, post-anti-cancer therapy. (A) Change in serum LDH levels when pre-treatment levels were normalized to one. (B) Serum LDH levels did not correlate significantly with serum fortilin levels.

FIG. 4. Known serum markers of apoptosis do not increase as much as fortilin levels upon anti-cancer therapy. Abbreviations: Cyt C; cytochrome c; n-DNA, nucleosomal DNA; fCK-18, fragmented cytokeratin-18; *, P<0.05; PRE, pre-anti-cancer therapy; POST, post-anti-cancer therapy; Δ, fold change. (A) Changes in serum cytochrome c (Cyt C) levels when their pre-treatment levels were normalized to one. There was no statistically significant association between serum Cyt C and fortilin levels by regression analysis (P=0.635, R2 =1.5%). (B) Changes in serum nucleosomal DNA (n-DNA) levels when their pre-treatment levels were normalized to one. There was no statistically significant association between serum n-DNA and fortilin levels by regression analysis (P=0.18, R₂=11.9%). (C) Changes in serum fragmented cytokeratin-18 (fCK-18) levels when their pre-treatment levels were normalized to one. There was no statistically significant association between serum fCK-18 and fortilin levels by regression analysis (P=0.082, R₂=18.8%).

FIG. 5. Serum fortilin levels increase in response to Jo2-antibody-induced apoptosis in the liver. Abbreviations: PBS, phosphate-buffered saline; CTL, control; i.p., intraperitoneal injection; ALT, alanine aminotransferase; n-DNA, nucleosomal DNA; H&E, hematoxylin and eosin staining; TUNEL, Terminal deoxynucleotidyl transferase dUTP nick-end labeling staining; *, P<0.05; **, P<0.01; ***, P<0.005. (A) C57BL/6J mice were injected with Jo2 antibody and sacrificed 5-9 hours later. (B) The livers of control and Jo2-treated mice. (C) Hematoxylin and eosin (H&E) and TUNEL staining of the control and Jo2-treated livers. Size bar: 100 μm. (D) TUNEL index (%) of the control and Jo2-treated livers. (E) Caspase-3 activities of control and Jo2-treated organs. (F) Serum alanine aminotransferase (ALT) levels in control and Jo2-treated mice. (G) Serum n-DNA levels in control and Jo2-treated mice. (H) Serum fortilin levels in control and Jo2-treated mice.

FIG. 6. Fortilin is released in the very early phase of apoptosis, before the compromise to the plasma membrane integrity. Abbreviations: α-Fas IgM, antihuman Fas IgM (clone CH11); CM, conditioned media; n-DNA, nucleosomal DNA; 7-AAD, 7-aminoactinomycin D; Cyt C, cytochrome c; fCK-18, fragmented cytokeratin-18; LDH, lactate dehydrogenase; PMΔ, plasma membrane change; *, P<0.05. (A) Experimental design. Jurkat cells were challenged by 12.5 ng/mL of anti-human Fas IgM (clone CH11) in Roswell Park Memorial Institute (RPMI) media with 5% fetal bovine serum (FBS). Cells and conditioned media were harvested at indicated time points and subjected to the respective sets of assays as described in the panel. 7-AAD cannot pass through the intact plasma membrane. 7-AAD cannot enter into the live cell. (B) 7-AAD staining to detect cells with change in plasma membrane integrity. (C) LDH concentrations in the conditioned media. (D) n-DNA concentration in the cell lysates that reflect the progression of apoptosis occurring within the cell. (E) Fortilin concentrations in the conditioned media. (F) Cyt C levels in the conditioned media. (G) n-DNA concentration in the conditioned media. (H) fCK-18 concentration in the conditioned media.

FIG. 7. Mouse fortilin levels do not differ between males and females. Abbreviations: NS, not statistically significant. Fortilin levels were determined for mouse serum samples from 12-week-old C57BL/6J male (N=15) and female (N=15) mice, using a newly developed fortilin ELISA.

FIG. 8. Human fortilin levels do not differ between males and females (A) or by age (B). Abbreviations: NS, not statistically significant. Fortilin levels were determined for 63 serum samples from apparently normal adult subjects, using a newly developed fortilin ELISA.

FIG. 9. 7-AAD staining of Jurkat cells stimulated by α-Fas IgM. Abbreviations: 7-AAD, 7-aminoactinomycin D. Size Bar: 200 μm. Jurkat cells were challenged by 12.5 ng/mL of anti-human Fas IgM (clone CH11) in Roswell Park Memorial Institute (RPMI) media with 5% fetal bovine serum (FBS). Cells were harvested at indicated time points and subjected to the 7-AAD staining 7-AAD cannot traverse through the intact plasma membrane. The cells with 7-AAD signal have disrupted plasma membrane. At least 200 cells were counted and the 7-ADD index was calculated as (the number of 7-AAD positive cells)/(the number of total cells)* 100.

FIG. 10. Jurkat cells do not secrete fortilin without anti-Fas IgM challenge. Abbreviations: NS, not statistically significant. 5×10⁵ Jurkat cells were seeded at each cell of 6-well plates using RPMI media supplemented by 5% FBS. The next day, cells were washed once with PBS and re-suspended in 1 mL of fresh RPMI media with 5% FBS. At times 0, 6, and 12 hours, 500 μL of cell suspension was harvested from each well in triplicates and subjected to centrifugation at 100 g for 5 min. The supernatant was transferred to a fresh microfuge tube and stored at −80° C. for fortilin ELISA.

FIG. 11. Comparison of fortilin with other apoptosis biomarkers and LDH. Abbreviations: Cyt C, cytochrome c; n-DNA, nucleosomal DNA; fCK-18, fragmented cytokeratin-18; LDH, lactate dehydrogenase. LDH is a cell death marker that is passively released through the damaged plasma membrane without apoptosis-specific modification. Although it is passively released from the cells unmodified, Cyt C can still be an apoptosis marker as Cyt C is released from the mitochondrial intermembrane space into the cytosol in the apoptosis-specific process. While it is passively released from the cells, fCK-18 is a caspase-cleaved product of the original cytoskeleton protein CK-18. Based on the data described in FIGS. 6F and 6H, both Cyt C and fCK-18 rely on the compromise in plasma membrane integrity for their release into extracellular space. n-DNA is an apoptosis-specific degradation product of nuclear DNA by the caspase-activated DNAse (CAD) and released before plasma membrane changes occur detectable by 7-AAD and LDH-release (FIG. 6G). Fortilin is unique because it does not undergo apoptosis-specific modification and is released in the very early phase of apoptosis, most likely via exosomes (FIG. 6E).

FIG. 12. Surface plasmon resonance with KeF1.RH1.H6 antibody immobilized, protein binding followed by sample (M03, M06, KeF2b.rb1.C12 and KeF2b.RE1.B4b antibodies).

FIG. 13. Surface plasmon resonance with M03 antibody immobilized, protein binding followed by sample (KeF2b.Rb1.C12, KeF2b.RE1.B4b and KeF1.RH1.H6 antibodies).

FIG. 14. Surface plasmon resonance with M06 antibody immobilized, protein binding followed by sample (KeF2b.Rb1.C12, KeF2b.RE1.B4b and KeF1.RH1.H6 antibodies).

FIG. 15. Surface plasmon resonance with KeF2.RA1 antibody immobilized, protein binding followed by sample KeF 1.RH1 and KeF 1.RA1 antibodies (preliminary data before sub cloning).

### DESCRIPTION

Fortilin (UniProt accession P13693), also known as translationally controlled tumor protein (TCTP) and IgE-dependent histamine releasing factor (HRF), is a 172-amino acid nuclear-cytosolic shuttle protein that was originally cloned in 1989 by Gross and others as a molecule abundantly expressed in tumor cells (Gross et al., Nucleic Acids Res, 17 (1989) 8367). A multifunctional protein implicated in various cellular functions (Kim et al., Arch Pharm Res, 23 (2000) 633-36; Graidist et al., Biochem J, 408 (2007) 181-91; Yarm, Mol Cell Biol, 22 (2002) 6209-21; Kashiwakura et al., J Clin Invest, (2011); Amzallag et al., J Biol Chem, 279 (2004) 46104-112; MacDonald et al., Science, 269 (1995) 688-90), fortilin possesses potent anti-apoptotic activity (Graidist et al., Biochem J, 408 (2007) 181-91; Fujita et al., FEBS Lett, 582 (2008) 1055-60; Graidist et al., J Biol Chem, (2004); Koide et al., Biochim Biophys Acta, (2009); Li et al., J Biol Chem, 276 (2001) 47542-49; Tulis et al., Circulation, 107 (2003) 98-105; Zhang et al., J Biol Chem, 277 (2002) 37430-38). It binds the sequence-specific DNA binding domain of p53 and prevents p53 from transcriptionally activating the proapoptotic gene Bax (Chen et al., J Biol Chem, (2011)). Fortilin also binds to and stabilizes MCL1 (Zhang et al., J Biol Chem, 277 (2002) 37430-38), a macrophage survival factor (Marriott et al., J Clin Invest, 115 (2005) 359-68; Steimer et al., Blood, 113 (2009) 2805-15). In addition to being intracellularly located, fortilin can be trafficked into exosomes—small secretory vesicles—and eventually be released into the extracellular space in an ER/Golgi-independent fashion (Amzallag et al., J Biol Chem, 279 (2004) 46104-12).

Since it is a potent anti-apoptotic molecule that can be secreted into the extracellular space, the inventors contemplate that fortilin is a serum apoptosis biomarker. As described herein, the inventors report that fortilin is present in the blood of healthy humans and mice. The inventors also show that anti-cancer chemotherapy or radiation therapy causes serum fortilin levels to increase, more robustly, sensitively, and specifically than fCK-18, Cyt C, or n-DNA in humans. The release of fortilin from the cell precedes any signs of compromised plasma membrane integrity. Serum fortilin is a sensitive and robust biomarker of apoptosis occurring in vivo.

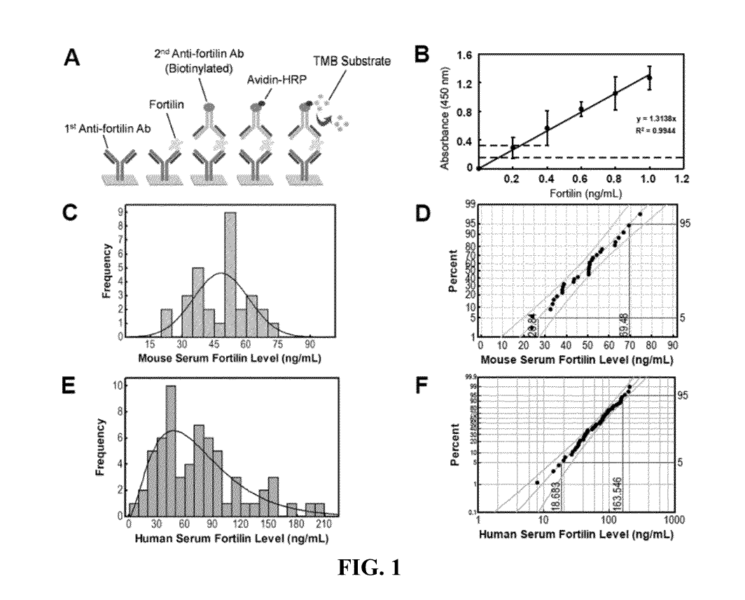

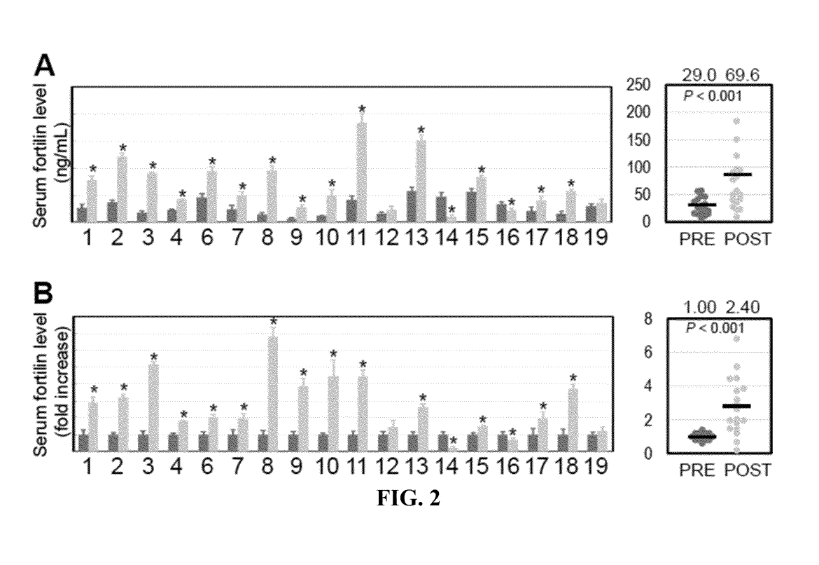

There are three innovations in the current work. First, the present study is the first to report the development and characterization of an ELISA system capable of measuring fortilin in mouse and human sera. The fortilin ELISA system both sensitively (detection limit=0.4 ng/mL) and reliably (% CV=8.6%) detected both human and mouse serum fortilins (FIG. 1). Second, it has been unknown whether fortilin circulates in the blood of normal subjects. The inventors are the first to show that fortilin circulates in the blood of normal congenic C57BL6/J mice and apparently healthy humans (FIG. 1). Third, using the ELISA system, the inventors demonstrate for the first time that serum fortilin is a unique biomarker of apoptosis (FIG. 2), distinct from and more sensitive and specific than the previously reported serum biomarkers of apoptosis, including Cyt C, n-DNA, and fCK-18 (FIG. 4, FIG. 11).

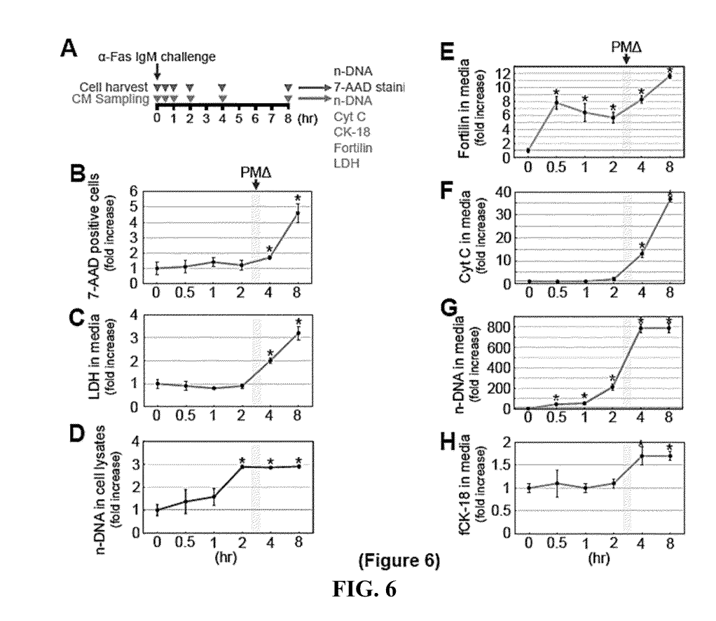

Renz and others reported that Cyt C not only translocates from the mitochondrial intermembrane space into the cytosol, but also is released into the extracellular space in response to apoptotic stimuli (Renz et al., Blood, 98 (2001) 1542-48). They found that the serum levels of Cyt C are elevated immediately after anti-cancer chemotherapy in patients with predominantly hematological malignancies (Renz et al., Blood, 98 (2001) 1542-48). When serum Cyt C levels were measured by quantitative Western blot analysis, more than a 2-fold increase in Cyt C levels was present in 8 out of 17 patients tested (47.1%) (Renz et al., Blood, 98 (2001) 1542-48). Other groups also reported that serum Cyt C levels are elevated in patients with un-treated malignant tumors, and that an elevated serum Cyt C level is an adverse prognostic marker (Osaka et al., J Cancer Res Clin Oncol, 135 (2009) 371-77; Osaka et al., International journal of laboratory hematology, 31 (2009) 307-14; Barczyk et al., Int J Cancer, 116 (2005) 167-73). However, Jemmerson and others reported that Cyt C release could occur from both apoptotic and necrotic cells (Jemmerson et al., Cell Death Differ, 9 (2002) 538-48). In addition, Osaka and others found that there was a significant and positive correlation between serum Cyt C and LDH levels (Osaka et al., J Cancer Res Clin Oncol, 135 (2009) 371-77). It is not entirely clear whether serum Cyt C becomes elevated solely and always due to apoptosis in vivo since, at least theoretically, any cell death stimuli that disrupt both mitochondria and the plasma membrane would release Cyt C into the extracellular space. In the current work, it was found that only 2 patients have statistically significant elevation of Cyt C levels (FIG. 4A) after anti-cancer therapy, while 14 (77.8%) out of 18 patients had statistically significant increase in fortilin levels (FIG. 4A). The relative lack of sensitivity of Cyt C, compared to fortilin, could be explained by the results of FIG. 6, where it was found that both Cyt C and fCK-18 require disruption of the plasma membrane to be released into the extracellular space (FIGS. 6F and 6H). On the contrary, fortilin and n-DNA did not require such plasma membrane changes and were released from the cells in the very early phase of apoptosis (on and after 0.5 hrs; FIGS. 6E and 6G), presumably through exosomes (Amzallag et al., J Biol Chem, 279 (2004) 46104-12) and apoptotic bodies (Turiak et al., Journal of proteomics, 74 (2011) 2025-33), respectively.

n-DNA are produced by the cleavage of chromosomal DNA by endonucleases activated during apoptosis and can be released into the circulation. Holdenrieder and others showed that patients with malignant tumors had higher levels of serum n-DNA than did healthy subjects and those with benign tumors and that serum n-DNA levels increased in response to anti-cancer therapy (Holdenrieder et al., Int J Cancer, 95 (2001) 114-20). Despite the fact that the generation of n-DNAs is specific to apoptosis and that they appear to be released from the cell in an active process via apoptotic bodies (Turiak et al., Journal of proteomics, 74 (2011) 2025-33) in the early phase of apoptosis (FIG. 6G), the serum n-DNA levels were found not to be as sensitive as serum fortilin levels in detecting apoptosis in the current cohort of patients—out of 17 samples tested, only 4 (23.5%) showed a statistically significant increase in n-DNA levels after anti-cancer therapy, while 2 (11.7%) had a statistically significant decrease (FIG. 4B). The apparently low sensitivity of n-DNA in human samples (FIG. 4B) may at least partly be due to the fact that circulating n-DNA is rapidly degraded by serum DNases within hours of its release into the circulation (Tamkovich et al., Ann N Y Acad Sci, 1075 (2006) 191-96).

Cytokeratin-18 (CK-18) is a major component of intermediate filaments of epithelial cells and tumors derived from such cells (Caulin et al., J Cell Biol, 138 (1997) 1379-94). During apoptosis, caspase-3 cleaves CK-18 into three fragments—NH₂-terminal, 26-kD; middle, 19-kD; and COOH-terminal, 3-kD. The M30 antibody recognizes a neo-epitope on the middle 19-kD fragment, exposed only after caspase-3 cleavage of CK-18 (Leers et al., The Journal of pathology, 187 (1999) 567-72). Levels of the caspase-cleaved, 19-kDa, CK-18 fragment (fCK-18) were reported to be significantly elevated in patients with gastric cancer versus healthy subjects (Oyama et al., Clinical and experimental medicine, 13 (2013) 289-95; Yaman et al., International immunopharmacology, 10 (2010) 719-22). fCK-18 levels also increased significantly after cancer chemotherapy (Bilici et al., Tumour biology, 33 (2012) 2201-08). However, it was also reported that both caspase-cleaved and uncleaved/intact CK-18s are elevated (a) in patients with cancer compared to healthy subjects (Ulukaya et al., Lung cancer, 56 (2007) 399-404; Yaman et al., International immunopharmacology, 10 (2010) 719-22) and (b) after cancer chemotherapy (Barczyk et al., Int J Cancer, 116 (2005) 167-73; Demiray et al., Cancer investigation, 24 (2006) 669-76), suggesting that the release of CK-18s relies on the disruption of the plasma membrane. In addition, CK-18 is present only in epithelial cells and may not be useful for the detection of apoptosis in other tissue types such as muscle, connective, and nervous tissues. In the current work, of 17 samples tested, only 2 (11.7%) showed a statistically significant increase in fCK-18 levels after anti-cancer therapy, while one (5.8%) had a statistically significant decrease (FIG. 4C), suggesting that serum fCK-18 levels are not as sensitive as serum fortilin levels in detecting apoptosis at least in the current cohort of patients with SCC undergoing anti-cancer therapy.

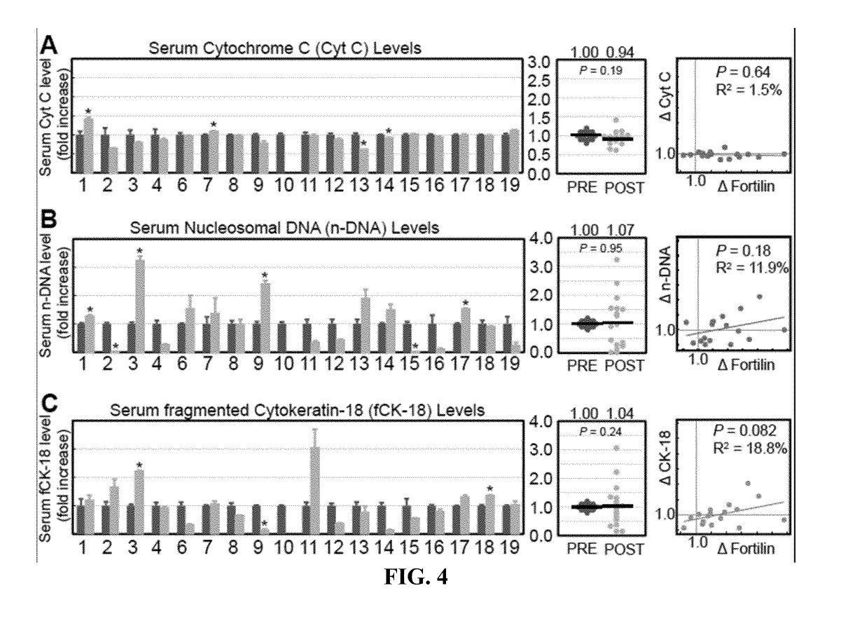

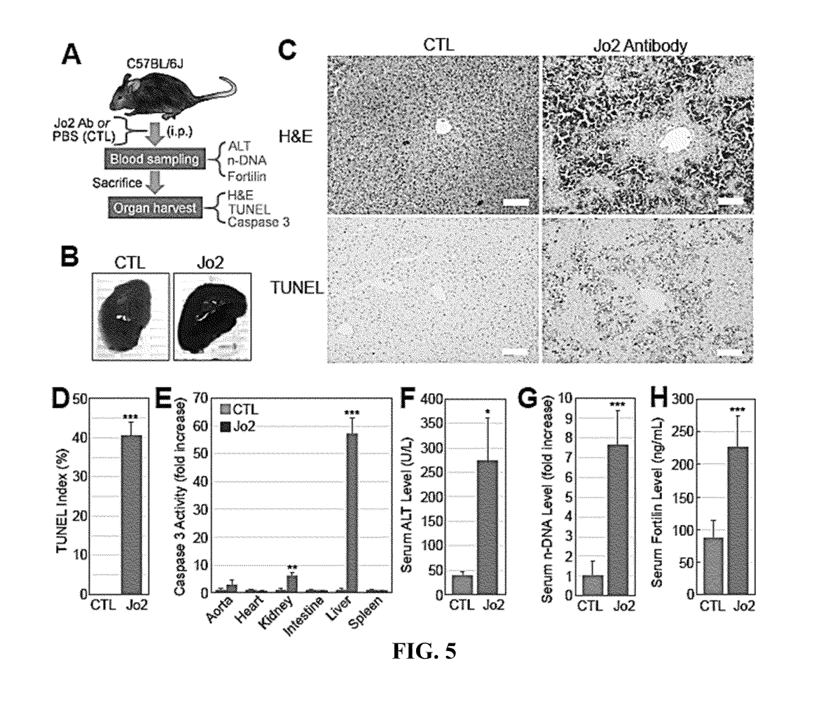

Fortilin has three key attributes that make it a preferred serum biomarker of apoptosis. First, it is released in the very early phase of apoptosis, well before the integrity of the plasma membrane is compromised (FIGS. 6B, 6C, and 6E). In other words, fortilin is an authentic apoptosis marker, and its release into the extracellular space does not require plasma membrane damage, unlike Cyt C, fCK-18, and LDH (FIGS. 6C, 6F and 6H). Second, it is detected in patients undergoing anti-cancer therapy more sensitively and consistently than are Cyt C, n-DNA, and fCK-18 (FIGS. 2 and 4, Table 1), most likely due to its stability in the blood and its ability to be secreted from the cell without plasma membrane disruption. Third, our clinical study (FIGS. 2, 3, and 4), animal experiments (FIG. 5), and cell-based experiments (FIG. 6) all support fortilin as a serum apoptosis biomarker (FIG. 11) superior to those previously reported.

TABLE 1
Summary of Patients Undergoing Anti-Cancer Chemotherapy

| | | | Diagnosis | | | | Therapy | |
|---|---|---|---|---|---|---|---|---|
| Patient # | Age | Sex | Type | Location | Recurrent | Stage | Radiation | Chemo |
| 1 | 49 | F | SCC | Cervical | Yes | IIIB | NO | Cisplatin |
| 2 | 35 | F | SCC | Cervical | Yes | IIIB | NO | Cisplatin |
| 3 | 40 | F | SCC | Cervical | Yes | IIIB | NO | Cisplatin |
| 4 | 48 | F | SCC | Cervical | No | IIIB | YES | Cisplatin |
| 6 | 49 | F | SCC | Cervical | No | IIIB | YES | Cisplatin |
| 7 | 53 | F | SCC | Cervical | No | IIB | YES | Cisplatin |
| 8 | 47 | F | SCC | Cervical | No | IV | YES | Cisplatin |
| 9 | 43 | F | SCC | Cervical | No | IIIB | YES | NO |
| 10 | 48 | M | SCC | Retromolar trigone | No | T4bN2b | YES | Cisplatin |
| 11 | 62 | M | SCC | Hypopharynx | No | T3N2 | YES | Cisplatin |
| 12 | 71 | M | SCC | Larynx & NSCLC | No | T3N2 & T2N0 | YES | NO |
| 13 | 38 | M | ACC | Maxillary sinus | No | NA | YES | NO |
| 14 | 51 | F | SCC | Tonsil | No | T4aN1 | YES | TPF |
| 15 | 59 | M | SCC | Retromolar trigone | No | T4BbN2b | YES | Cisplatin |
| 16 | 53 | F | SCC | Base of the tongue | No | T4N2 | YES | NO |
| 17 | 35 | M | Mucoepidermoid | Base of the tongue | No | IV | YES | NO |
| | | | carcinoma | | | | | |
| 18 | 67 | M | SCC | Base of the tongue | No | T4bN2b | YES | Cisplatin |
| 19 | 52 | M | SCC | Tonsil | No | IVa(T3N2) | YES | Cisplatin |

| | Post-Treatment | | | Established apotosis | | | |
|---|---|---|---|---|---|---|---|
| | sampling (days | | | biomarkers | | | |
| Patient # | [symbol] | LDH | Fortilin | Anyone | Cyt C | n-DNA | CK-18 |
| 1 | 26 | | ↑ | ↑↑ | ↑ | ↑ | |
| 2 | 11 | | | | | | |
| 3 | 50 | | ↑ | ↑↑ | | ↑ | ↑ |
| 4 | 16 | ↑ | ↑ | | | | |
| 6 | 3 | | ↑ | | | | |
| 7 | 7 | | ↑ | ↑ | ↑ | | |
| 8 | 1 | | | | | | |
| 9 | 7 | | ↑ | ↑ | | ↑ | |
| 10 | 20 | | ↑ | | | | |
| 11 | 44 | | | | | | |
| 12 | 1 | ↑ | ↑ | | | ↑ | |
| 13 | 1 | | ↑ | | | | |
| 14 | 54 | | ↑ | | | | |
| 15 | 7 | | ↑ | | | | |
| 16 | 1 | | ↑ | | | | |
| 17 | 1 | | ↑ | ↑ | | ↑ | |
| 18 | 1 | | ↑ | ↑ | | | ↑ |
| 19 | 16 | | | | | | |

Abbreviation:
F, female;
M, male;
SCC, squamous cell carcinoma;
ACC, adenocystic carcinoma;
NSCLC, non-small-cell lung carcinoma;
TPF, docetaxel, cisplatin, 5-fluorouracil;
NA, data not available.
Patient #10: a sufficient amount of post-treatment sample available only for fortilin ELISA.
Patient #5: excluded from the study as post-treatment sample volume was not sufficient for any assays. [symbol]
indicates data missing or illegible when filed

In conclusion, the inventor for the first time establishes serum fortilin as a viable apoptosis biomarker, which is secreted from apoptosing cells into the circulation in the very early phase of apoptosis and detectable by the ELISA.

### I. IMMUNOASSAYS

Detecting and/or quantifying polypeptide(s) may be performed using an immunological method, involving an antibody, or a fragment thereof capable of specific binding to the polypeptide. In certain aspects the polypeptide is fortilin. Suitable immunological methods include sandwich immunoassays, such as sandwich ELISA, in which the detection of polypeptide(s) is performed using two antibodies which recognize different epitopes on a polypeptide(s); radioimmunoassays (RIA); direct, indirect or competitive enzyme linked immunosorbent assays (ELISA); enzyme immunoassays (EIA); Fluorescence immunoassays (FIA); western blotting; immunoprecipitation; and any particle-based immunoassay (e.g. using gold, silver, or latex particles, magnetic particles, or Q-dots). Immunological methods may be performed, for example, in microtitre plate or strip format.

Immunological methods in accordance with the invention may be based, for example, on any of the following methods.

Immunoprecipitation is the simplest immunoassay method; this measures the quantity of precipitate, which forms after the reagent antibody has incubated with the sample and reacted with the target antigen present therein to form an insoluble aggregate. Immunoprecipitation reactions may be qualitative or quantitative.

In particle immunoassays, several antibodies are linked to the particle, and the particle is able to bind many antigen molecules simultaneously. This greatly accelerates the speed of the visible reaction. This allows rapid and sensitive detection of the biomarker.

Radioimmunoassay (RIA) methods employ radioactive isotopes such as 1¹²⁵ to label either the antigen or antibody. The isotope used emits gamma rays, which are usually measured following removal of unbound (free) radiolabel. The major advantages of RIA, compared with other immunoassays, are higher sensitivity, easy signal detection, and well-established, rapid assays. The major disadvantages are the health and safety risks posed by the use of radiation and the time and expense associated with maintaining a licensed radiation safety and disposal program. For this reason, RIA has been largely replaced in routine clinical laboratory practice by enzyme immunoassays.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA). ELISA methods may use two antibodies one of which is specific for the target antigen and the other of which is coupled to an enzyme, addition of the substrate for the enzyme results in production of a chemiluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of polypeptide(s) as described herein.

The Biotin-Avidin or Biotin-Streptavidin systems are generic labeling systems that can be adapted for use in immunological methods described herein. One binding partner (hapten, antigen, ligand, aptamer, antibody, enzyme etc) is labeled with biotin and the other partner (surface, e.g. well, bead, sensor etc.) is labeled with avidin or streptavidin. This is conventional technology for immunoassays, gene probe assays, and (bio)sensors, but is an indirect immobilization route rather than a direct one. For example a biotinylated ligand (e.g., antibody or aptamer) specific for a peptide biomarker of the invention may be immobilized on an avidin or streptavidin surface, the immobilized ligand may then be exposed to a sample containing or suspected of containing a target polypeptide(s) in order to detect and/or quantify a polypeptide(s). Detection and/or quantification of the immobilized antigen may then be performed by an immunological method as described herein.

A kit for diagnosing or monitoring a subject is also provided. In certain aspects the subject is a cancer patient. In a further aspect the subject is a cancer patient undergoing cancer treatment, such as chemotherapy or radiation therapy. Suitably a kit according to the invention may contain one or more components selected from the group: a ligand specific for polypeptide(s) (e.g., fortilin), one or more controls, one or more reagents and one or more consumables; optionally together with instructions for use of the kit in accordance with any of the methods defined herein.

### II. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Detecting Fortilin Levels in Serum

A. Materials and Methods

Cell Culture and Cell Lines. The Jurkat cell lines (Clone E6-1) were purchased from American Type Culture Collection (ATCC, Manassas, Va.). All cell lines were maintained in high-glucose Roswell Park Memorial Institute (RPMI) medium supplemented with 10% fetal bovine serum (FBS) at 37° C. in an atmosphere containing 10% CO₂.

Cell-based Assay of Biomarkers Release and Plasma Membrane Disruption. Jurkat cells were seeded in 18 wells of 6-well plates (5×10⁵ cells/well) in RPMI medium with 5% FBS. The next day, the cells were washed once with phosphate buffered saline (PBS) and re-suspended in 1 mL of RPMI medium containing 12.5 ng/mL of antihuman Fas IgM (clone CH11). At each time point (0, 0.5, 1, 2, 4, and 8 hr, N=3 for each time point), 500 μL of cell suspension was harvested and centrifuged at 100 g for 5 min, transferred the medium to fresh microfuge tubes, and froze both the media and cell pellets separately at −80° C. until the assays for LDH, n-DNA, fortilin, Cyt C, and fCK-18, were performed. For 7-AAD staining, 20 μL of 7-AAD solution (BD Pharmingen) was added to 400 μL of cell suspension and incubated for 10 min at room temperature, shielded from light. Total and 7-AAD-positive cells were counted under the fluorescence microscope as described previously (Cerne et al., Protoplasma, 250 (2013) 1131-37). The integrity of the plasma membrane of the cells with positive 7-AAD signal is compromised. At least 200 cells were counted and the 7-ADD index was calculated as (the number of 7-AAD-positive cells)/(the number of total cells)*100.

Mouse model of targeted liver apoptosis: Induction of liver-specific apoptosis. All animal procedures were performed according to a protocol approved by the UTMB Institutional Animal Care and Use Committee (IACUC), in accordance with the National Institutes of Health guidelines and the “Position of the American Heart Association on Research Animal Use.” Apoptosis was induced in the liver of C57BL/6J male mice (12 weeks of age) by intraperitoneal administration of the Jo2 anti-Fas antibody (1.25 μg/body weight in grams, resulting in approximately 25 μg of antibody per mouse): PBS was used as a control. Once injected, the mice became ill within 3 hours; half of them were dead within 6 hours, as described previously (Ogasawara et al., Nature, 364 (1993) 806-09). At 5-9 hours after anti-Fas injection, when they were clinically moribund, the mice were sacrificed, their blood collected, and the organs harvested for further analyses. Jo2 antibody binds the mouse Fas antigen and induces Fas-mediated apoptosis in the liver without affecting any other tissues as reported previously (Ogasawara et al., Nature, 364 (1993) 806-09). Transmission electron microscopic examination reportedly showed a lack of phagocytosis of apoptosed cells. Also, there was no gross leakage of cell contents into the extracellular space observed by the same examination (Ogasawara et al., Nature, 364 (1993) 806-09).

Caspase-3 Activity. Caspase-3 assays were performed as described previously (Koide et al., Biochim Biophys Acta, 1790 (2009) 326-38). In brief, cells were suspended in cell lysis buffer (10 mM Tris, pH 7.5, 100 mM NaCl, 1 mM EDTA, 0.01% Triton X-100), subjected to three freeze-thaw cycles, and centrifuged at 14,000 g. Aliquots of cleared cell lysates were incubated with 2.5 mM rhodamine 110 bis-(N-CBZL-aspartyl-L-glutamyl-L-valyl-L-aspartic acid amide)(Z-DEVD-R110) substrate (Invitrogen-Molecular Probes, Grand Island, N.Y.). Caspase-3 activities were determined every 5 min for 90 min by measuring fluorescence (excitation/emission=496/520 nm), using the SpectraMax Plus microplate spectrophotometer (Molecular Devices, Sunnyvale, Calif.), and expressed as relative fluorescence units (RFU).

TUNEL staining. TUNEL staining was performed as previously described (Chen et al., J Biol Chem, (2011)), using the FragEL™ DNA Fragmentation Detection Kit (EMD Millipore, Calbiochem, Billerica, Mass.), according to the manufacturer's instructions. At least 600 cells were counted and TUNEL indices were calculated as the number of TUNEL-positive cells divided by the number of total cells counted and expressed as percentages.

Lactate dehydrogenase (LDH) activity assay. Serum LDH activity was measured by the LDH Activity Assay Kit (Sigma-Aldrich, St. Louis, Mo.; Catalog Number: MAK066) according to the manufacturer's instructions.

Serum Cyt C assay. Serum Cyt C was quantified by the Human Cytochrome C Quantikine ELISA kit (R&D Systems, Minneapolis, Minn.; Catalog Number: DCTC0) according to the manufacturer's instructions.

Serum alanine transaminase (ALT) assay. Serum ALT was quantified by the mammalian liver profile rotor and VETSCAN VS2 (Abaxis, Union City, Calif.) according to the manufacturer's instructions.

DNA fragmentation assay. The Cell Death Detection ELISA PLUS kit (Roche, Indianapolis, Ind., Catalog Number:11774425001) was used according to the manufacturer's instruction and modifications described previously (Chen et al., J Biol Chem, (2011)). 20 μL of serum from a patient was added to a well of a streptavidin-coated 96-well plate, in triplicate. 80 μL of incubation buffer (PBS supplemented with 1% BSA, 0.5% Tween 20 and 1 mM EDTA) containing peroxidase-conjugated mouse anti-DNA antibody (MCA-33) and mouse biotinylated anti-histone antibody (H11-4) was then added to the wells and incubated the plate for 2 hours at room temperature on a shaker. The plate was then washed three (3) times with incubation buffer before we added 100 μl of 2,2′-Azinobis[3-ethylbenzothiazoline-6-sulfonic acid]-diammonium salt (ATBS) solution to each well, incubated at room temperature on a shaker (300 rpm) until the color development was sufficient for reading (2-10 min), and added 100 μl of the ATBS Stop Solution.

Fragmented cytokeratin-18 (fCK-18) determination. Cytokeratin-18 (CK-18), a 48-kDa, 423 amino-acid polypeptide, is a major component of intermediate filaments of cells of epithelial origin. M30 is a monoclonal antibody produced by immunizing Balb/c mice with two purified CK18 fragments (Leers et al., The Journal of pathology, 187 (1999) 567-72). The epitope of M30 is the 387-396th amino acids of CK18 (EDFNLGDALD), representing the COOH-terminal amino acid residues of the intermediate 19-kDa fragment generated by caspase-3 and caspase-7. M30 does not recognize the intact CK18 where it has not been cleaved between 396th asparatic acid (D) and 397th serine (S) by caspases (Leers et al., The Journal of pathology, 187 (1999) 567-72), thus making M30 reactivity specific for apoptosis. The exact molecular mechanism by which CK-18 is released from epithelial cells undergoing apoptosis remains unknown (John et al., Cell death & disease, 4 (2013) e886). Quantification of the caspase-generated neoepitope of CK-18 in serum samples was performed using the M30-Apoptosense ELISA kit (Peviva, Bromma, Sweden) as described previously and according to the manufacturer's instructions, in triplicates. The standard curves were generated by using synthetic immunogenic peptides provided with the kit.

ELISA to quantify the serum concentration of mouse and human fortilin. A polystyrene 96-well plate (BD Falcon, Bedford, Mass.) was coated with 50 μL, of capture anti-fortilin antibody (Abnova, Taipei City, Taiwan) diluted at 1 μg/mL in PBS and incubated at 4° C. overnight. After the wells were washed five (5) times with wash buffer (PBS with 0.1% Tween 20), they were blocked with 100 μL, of blocking buffer (PBS with 1% BSA) for 1 hour at room temperature. The wells were then washed five (5) times with wash buffer. 100 μL, each of the samples, dissolved at an appropriate concentration in dilution buffer (PBS with 0.1% BSA), was then added to each well and incubated at 37° C. for 3 hours. The wells were then washed five (5) times with wash buffer. 100 μL of biotinylated anti-fortilin detection antibody (Abnova) diluted at 1 μg/mL in dilution buffer was added to each well, and the plate was incubated at 37° C. for 2 hours. After wells were washed five (5) times with wash buffer, 100 μL, of avidin-HRP (eBioscience, San Diego, Calif., diluted at 1:500 in dilution buffer) was added to each well. The wells were then washed five (5) times with wash buffer. To detect bound antibody, 100 μL, of Strep™Ultra TMB-ELISA (Thermo Fisher Scientific, Waltham, Mass.) was added to each well and the plate was incubated at room temperature for 30 min before 50 μL of 2 M sulfuric acid was added to stop the reaction. The signal was read with a plate reader at 450 nm. The Coefficient of Variation (CV) was determined as % CV=SD/mean×100 using the data obtained from normal human subjects. The limit of detection was defined as the fortilin concentration at which the current ELISA system gives a statistically significant value above that of the zero analyte (dilution buffer alone) at a 99% confidence level, i.e., the means of the quadruplicates of the zero analyte and those of fortilin at the detection-limit concentration must differ by three SDs.

Collection of human serum samples. Human sera were collected under protocols approved by the UTMB Internal Review Board. Detailed information on the samples from the patients undergoing anti-cancer therapy is shown in Table 1 above. Written informed consent was received from participants prior to sample collection. The samples were frozen at −80° C. until the described assays were performed. All samples were free of any identifying information at the time of assay.

Statistical Analysis. The degree of spread of the data was expressed as the standard deviation (±SD). P<0.05 was considered to be statistically significant. The Student's t-test was employed for comparing the means of two groups.

B. Results

Fortilin ELISA development. There have been no robust quantitative assays reported for fortilin. To evaluate fortilin as a serum apoptosis biomarker, a sandwich enzyme-linked immunosorbent assay (ELISA) of fortilin was developed using two distinct anti-fortilin monoclonal antibodies and avidin-based signal amplification as described in detail in the Materials and Methods section (FIG. 1A). The detection limit of the developed ELISA was 0.4 ng/mL (FIG. 1B) with an 8.6% coefficient of variation (CV).

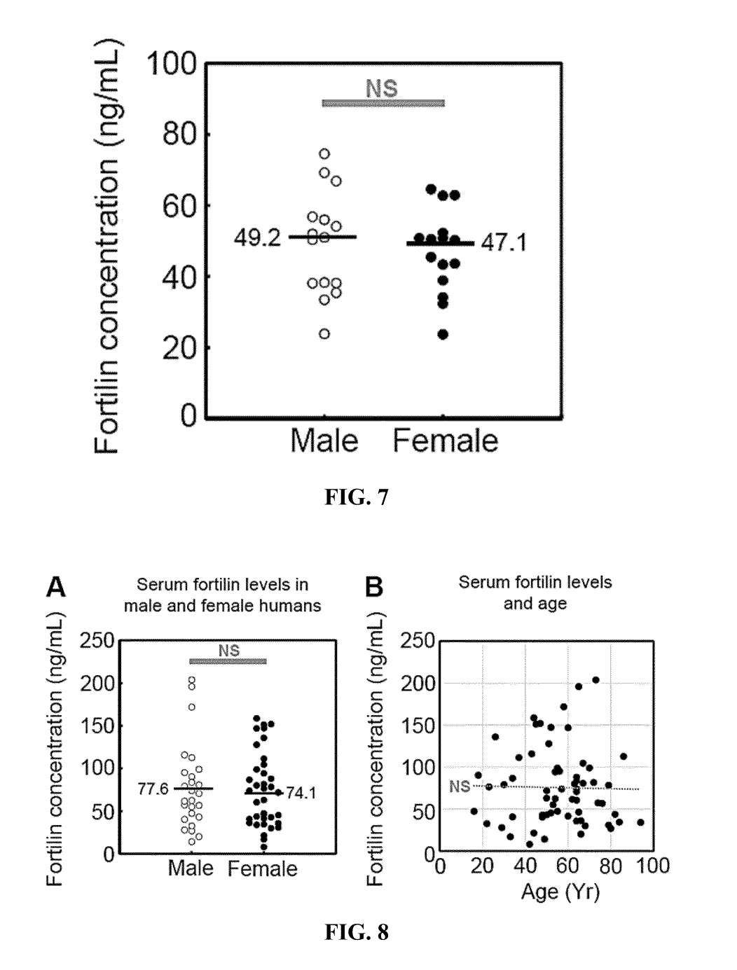

Fortilin circulates in the blood of normal humans and mice. To test the hypothesis that fortilin circulates in the blood, the sera from 12-week-old C57BL/6J male and female mice (n=30) were subjected to the above fortilin ELISA assay, showing average mouse serum fortilin levels of 48.16±12.96 ng/mL with no significant difference between male and female mice (males vs. females=49.22±14.48 vs. 47.10±11.66 ng/mL, n=15 each, P=0.663; FIG. 7). The samples were distributed normally (Anderson-Darling [AD] normality test, AD value=0.292, P=0.581; FIG. 1C) with the 5 and 95 percentile values of 28.84 and 69.48 ng/mL (FIG. 1D). The same ELISA assay was used to examine the sera from 63 patients presenting to clinic for routine examination. Their average fortilin serum concentration was 75.57±45.79 ng/mL with no significant difference between male and female human subjects (males vs. females=77.6±49.9 vs. 74.1±43.3 ng/mL, n=26 and 37, respectively, P=0.77; FIG. 8A). There was also no statistically significant correlation between age and fortilin levels (FIG. 8B). Unlike the mouse samples, the human samples did not distribute normally (Anderson-Darling [AD] normality test, P<0.005; FIG. 1E). The data best fits the gamma distribution with the lowest AD value (0.182, P>0.250), with the 5 and 95 percentile values of 18.68 and 153.55 ng/mL (FIG. 1F). Taken together, these data suggest that fortilin circulates in the blood of both normal humans and mice.

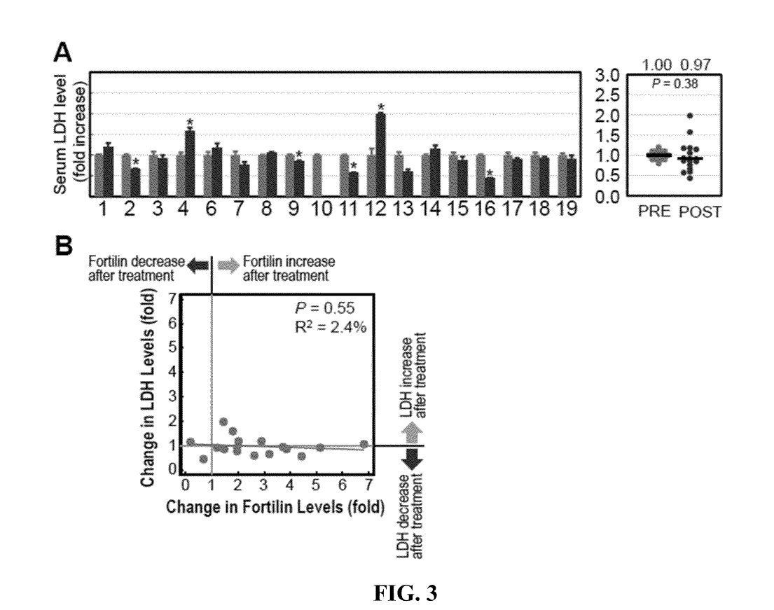

Serum fortilin levels are significantly elevated after anti-cancer chemotherapy and radiation therapy. Fortilin has been shown to protect cells against apoptosis and to be released into the extracellular space via secretory exosomes (Amzallag et al., J Biol Chem, 279 (2004) 46104-12). To test the hypothesis that serum fortilin levels reflect apoptosis occurring at the tissue level, serum fortilin concentrations were quantified in pre- and post-treatment sera of patients with solid malignancies (cervical/neck cancers and squamous cell carcinoma [SCC]) who were undergoing anticancer chemo- or radiation therapy (“anti-cancer therapy” hereafter), as both anticancer drugs and ionizing radiation trigger apoptosis in cancer tissue (Table 1). The mean pre-treatment serum fortilin levels in these cancer patients was 29.0±15.5 ng/mL. After chemotherapy, however, the mean serum fortilin levels increased 2.40 fold to 69.6±47.2 ng/mL (P<0.001, Paired Student's t-test; FIG. 2). Of 18 samples tested, 14 (77.8%) showed a statistically significant increase in fortilin levels after anti-cancer therapy while two (11.1%) were significantly decreased (FIG. 2). These data indicate that serum fortilin levels generally increase in response to anti-cancer therapy. The inventors also determined the lactate dehydrogenase (LDH) levels in these samples as these levels may reflect anti-cancer-therapy-induced tissue necrosis (Renz et al., Blood, 98 (2001) 1542-48). The mean pretreatment serum LDH level was 33.36±12.54 mU/mL, while the mean post-treatment serum LDH level was 30.46±11.81 IU/mL (P=0.38, NS). Out of 17 samples tested (the patient #10 was excluded as we did not have enough post-treatment sera remaining for assays), only two (11.8%) had a statistically significant increase in LDH levels after anti-cancer therapy while four (23.5%) were significantly decreased (FIG. 3A). Further, regression analysis showed no statistically significant association between the fold-change of serum fortilin levels and that of LDH levels after anti-cancer therapy (P=0.552, R2=2.4%; FIG. 3B). These data suggest that anti-cancer treatment induced no significant degree of tissue necrosis and that fortilin elevation was not due to the necrotic plasma membrane damage in cancer cells (FIGS. 3A and 3B).

Serum fortilin is superior to the other apoptosis biomarkers. To compare serum fortilin levels with the other apoptosis biomarkers described in the literature, the same samples were assayed for Cyt C, n-DNA, and fCK-18 levels.

First, it was found that the mean serum Cyt C levels did not statistically change (from 0.90±0.26 to 0.83±0.31 ng/μL, P=0.19) between before and after anti-cancer therapy. Out of 17 samples tested, only two (11.7%) showed a statistically significant increase in Cyt C levels after anticancer therapy while two (11.7%) had a statistically significant decrease (FIG. 4A).

Next, serum n-DNA levels were determined before and after anti-cancer therapy and found that the mean serum n-DNA levels did not statistically change (from 0.23±0.25 to 0.21±0.38 ng/μL, paired Student's t-test, P=0.820). Of 17 samples tested, only 4 (23.5%) showed a statistically significant increase in n-DNA levels after anti-cancer therapy, while 2 (11.7%) had a statistically significant decrease (FIG. 4B).

Upon assaying serum fCK-18 levels before and after anti-cancer therapy, it was found that the mean serum fCK-18 levels did not statistically change (from 139.78±98.1 to 148.4±100.3 U/L, paired Student's t-test, P=0.197). Of 17 samples tested, only 2 (11.7%) showed a statistically significant increase in fCK-18 levels after anti-cancer therapy while one (5.8%) had a statistically significant decrease (FIG. 4C).

Serum fortilin levels were significantly elevated in all patients who had at least one of three established biomarkers of apoptosis (Cyt C, n-DNA, or fCK-18) elevated (Patients #1, 3, 7, 9, 17, and 18) (Table 1). They were not elevated in patients whose established biomarkers were all negative (Patients #12 and 19) (Table 1). These data presented above, when taken together, suggest that the elevation of serum fortilin levels accurately predicts apoptosis occurring in vivo and that serum fortilin levels more sensitively detect in vivo apoptosis than do Cyt C, n-DNA or fCK-18 levels.

Serum fortilin levels are drastically elevated in mice with apoptosis-induced liver damage. To further validate the serum fortilin level as a biomarker of apoptosis in vivo, a mouse model of apoptosis-induced liver damage was used in which intraperitoneally administered Armenian hamster anti-Fas-antigen antibody (Jo2) rapidly and selectively induces Fas-mediated hepatocyte apoptosis, leading to severe liver damage without affecting other organs (Ogasawara et al., Nature, 364 (1993) 806-09). Since different strains of mice exhibit a vastly different response to Jo2 antibody (Kakinuma et al., Toxicol Pathol, 27 (1999) 412-20), the liver injury in C57BL/6J mice to which were intraperitoneally administered Jo2 antibody were characterized (FIG. 5A). Within 3 hours of injection, mice became grossly ill and were dead within 9 hours. The livers of mice treated with Jo2 antibody were hemorrhagic, both grossly and at a microscopic level, resembling human fulminant hepatitis (FIG. 5B and FIGS. 5C-H&E). TUNEL staining (FIG. 5C-TUNEL, FIG. 5D) showed numerous TUNEL-positive, apoptotic hepatocytes suggesting that fas-mediated hepatocyte apoptosis predominantly resulted in liver damage in the current system (TUNEL indices: control (CTL) vs. Jo2=0.00±0.00 vs. 40.46±3.46%, n=5, P<0.005). The vast majority of Jo2 antibody's effects were on the liver, as the caspase 3 activity of the liver increased 55.33-fold while the kidney, the second most severely affected organ, showed only a 6.09-fold increase in caspase-3 activity (P<0.005, n=3; FIG. 5E). As expected, the administration of Jo2 antibody resulted in a drastic increase in the serum alanine transaminase (ALT) levels (control vs. Jo2=39.33±7.02 vs. 272.50±88.39 U/L, n=3, P<0.05; FIG. 5F). Serum n-DNA levels were also elevated in Jo2-treated animals (control vs. Jo2=1.00±0.75 vs. 7.64±1.75 A.U., n=5, P<0.005; FIG. 5G). In this system, serum fortilin levels were found to be 2.63-fold elevated in Jo-2 treated animals vs. their control (86.18±27.71 vs. 226.31±48.51 ng/mL, n=4, P<0.005; FIG. 5H). These findings in the well-characterized mouse model of apoptosis-induced liver injury suggest that serum fortilin levels represent a viable marker of in vivo apoptosis.

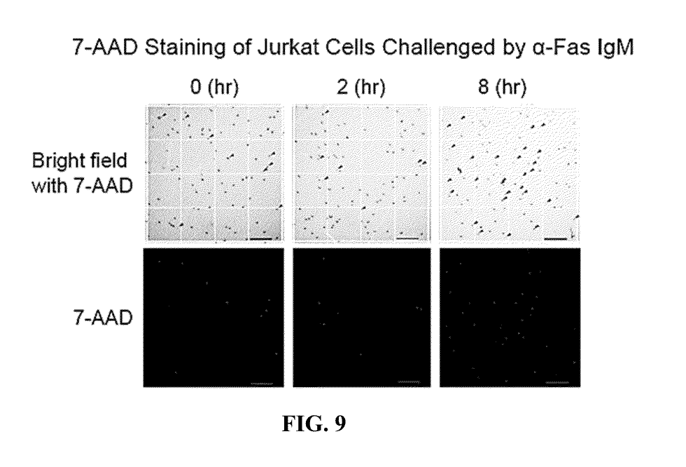

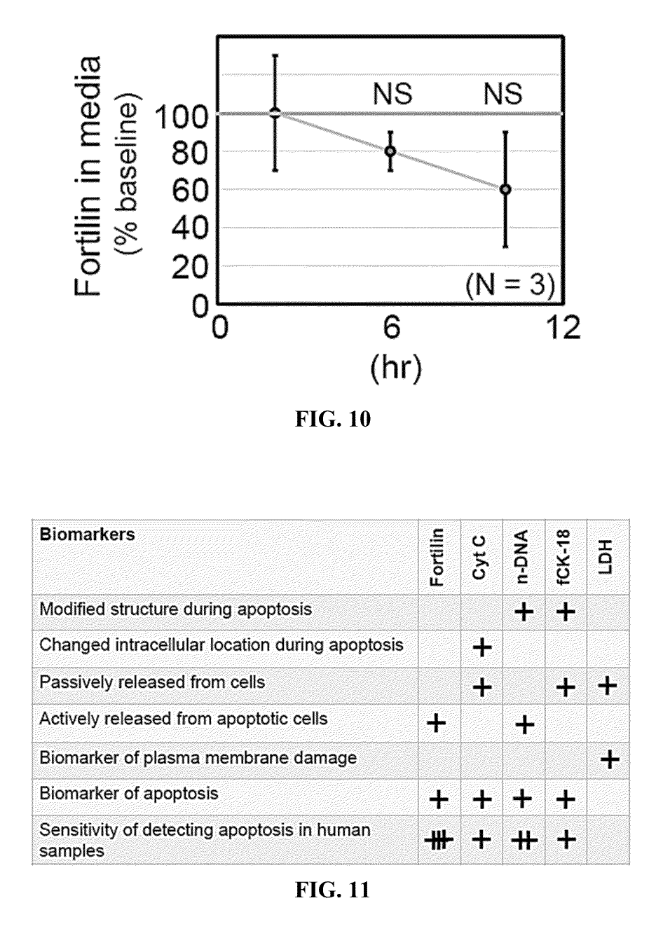

Fortilin excretion from the cell precedes the compromise of the plasma membrane integrity. To evaluate the possibility that the elevation of serum fortilin levels observed above was due solely to the passive release of fortilin from the damaged cells, Jurkat cells, a human T lymphocyte cell line, were challenged with anti-human Fas IgM (CH-11, 12.5 ng/mL), harvested aliquots of cells and conditioned media at times 0, 0.5, 1, 2, 4, and 8 hours (FIG. 6A), and subjected the cells to (a) 7-aminoactinomycin D (7-ADD) staining to identify cells with plasma membrane damage (FIG. 9) and (b) a DNA fragmentation assay to monitor the progression of apoptosis. The conditioned media was subjected to assays for n-DNA, Cyt C, fCK-18, LDH, and fortilin (FIG. 6A). In this system, 7-ADD-positive Jurkat cells did not significantly increase until 4 hours after the induction of apoptosis (FIG. 6B). Similarly, the LDH concentration in the media did not significantly increase until 4 hours after apoptosis was induced (FIG. 6C). Taken together, these data suggest that plasma membrane integrity becomes compromised (PMΔ) between 2 and 4 hours after the initiation of Fas-induced apoptosis in this system. DNA fragmentation within the cell began right after anti-Fas stimulation, progressed steadily, and was completed by 2 hours after apoptosis was induced as quantified by DNA fragmentation assays performed on the cell lysates (FIG. 6D), suggesting that plasma membrane disruption occurring between 2 and 4 hours after apoptosis induction coincided with the very late phase of apoptosis in this system. Using this system and fortilin ELISAs, we tested whether fortilin was actively released from apoptosing Jurkat cells before the plasma membrane disintegrated. Jurkat cells do not secrete fortilin into the media without apoptotic stimuli (FIG. 10). Strikingly, fortilin concentrations in the media drastically increased just 30 min after the induction of apoptosis, well before there was any detectable signs of plasma membrane disintegration (FIG. 6E; 0.5, 1, and 2 hours), supporting the idea that fortilin is actively released from the apoptosing cells via exosomes (Amzallag et al., J Biol Chem, 279 (2004) 46104-112). While n-DNA exhibited release kinetics similar to those of fortilin (FIG. 6G), the concentrations of Cyt C and fCK-18 in the medium did not increase until 4 hours had passed, suggesting that the release of those apoptosis biomarkers required the change to the plasma membrane (FIG. 6F and FIG. 6H). These data suggest that fortilin is actively released from apoptosing cells in the very early phase of apoptosis and its release does not require a compromise to the integrity of the plasma membrane detectable by the elevation of LDH and traversing of 7-AAD into the cells.

### Example 2

### Antibody Development

A. Methods

Immunisation and Serum Titre: Mice were immunised intraperitonealy 3 times at two-week intervals with a combination of 16 μg of antigen and an immune adjuvant (Sigma-Aldrich cat# S6322) in combination with methylated CpG. A serum sample was collected from the immunized mice and reactivity to the antigen was tested by ELISA at a dilution of 1:250 and 1:1250 and compared to a pre-immunization sample. The mouse with the highest titre was selected for fusion.

Hybridoma Fusion: To generate hybridoma cells the mouse spleen was excised, dissociated into a single cell suspension and fused to SP2/0-Ag14 myeloma cells using polyethylene glycol. The resultant hybridoma cells were grown in Azaserine Hypoxantine containing medium in 20×96 well tissue culture plates.

Screening: Hybridoma colonies were grown for 10 days at which point the number of hybridoma colonies was determined and after a further 3 days incubation an aliquot of antibody supernatant taken for screening. The supernatant was assayed for reactivity to the antigen and any screening samples, firstly by microarray followed by ELISA of any IgG microarray positive clones.

Expansion and Freezing: The highest responding ELISA positive clones were then expanded into a 24 well tissue culture plate for 3-4 days at which point they were expanded to a 6 well tissue culture plate. The cells were seeded at a 1:5 (supernatant wells) and 1:25 (cells wells) ratio. Once the cell wells reached 80% confluence the cells were extracted and frozen in liquid nitrogen in 10% DMSO and the supernatant from the supernatant wells was pooled and frozen at −20° C.

Subcloning: Clones selected for sub-cloning were subjected to at least 2 rounds of serial dilution. After each dilution stage, cells were grown for 4-5 days and single colonies producing antibody positive to the antigen were determined by supernatant ELISA and the top 2 clones were expanded for further rounds. The final monoclonal cell-lines were expanded into E-well cell-culture plates for 4-5 days, the supernatant was extracted and frozen down along with the cells.

Surface Plasmon Resonance: To determine a complementary pair, the top 10 binding antibodies (based on microarray and ELISA data) were purified from 2.5% serum culture media and analyzed for complementary binding to the Fortilin protein using Surface Plasmon Resonance (SPR). The experiment was performed using a ProteOn XPR36 SPRi biosensor equipped with GLH chips. The chips were conditioned with 0.5% SDS, 50 mM NaOH and 100 mM HCl. Following conditioning, the lanes to be used were activated using equal parts EDAC and NHS. The antibodies were immobilized onto the chip and the Fortilin protein was flown across to detect a binding event. Once Fortilin was bound to the immobilized antibody the set of antibodies were flown across the complex to determine if any of the antibodies bound to the Fortilin in a sandwich configuration.

B. Results

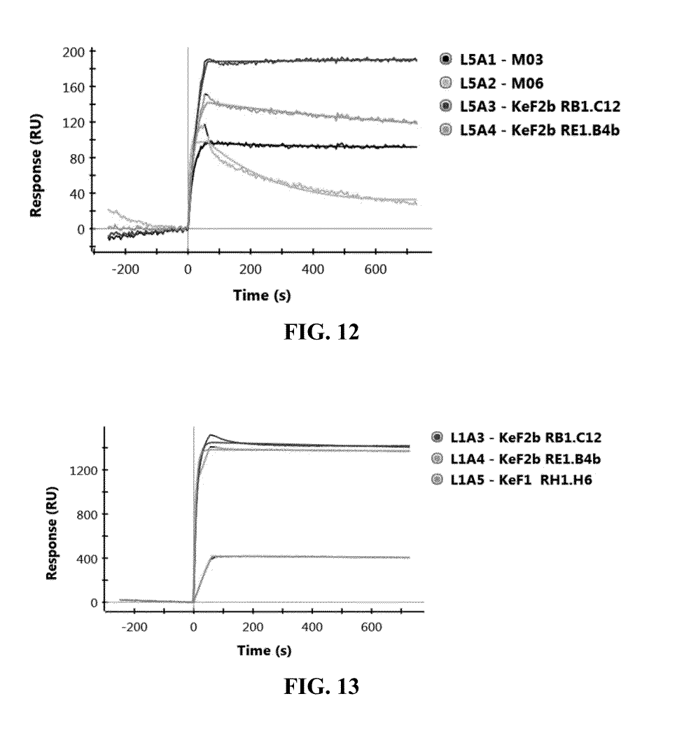

(1) KeF1.RH1.H6 immobilized, protein binding followed by sample (M03, M06, KeF2b.rb1.C12 and KeF2b.RE1.B4b)(FIG. 12)

| Param- | | Concen- | | | |
|---|---|---|---|---|---|
| eter | Sample | tration | ka | kd | KD |
| Unit | Name | M | 1/Ms | 1/s | M |
| L5A1 | M03 | 2.50E−07 | 2.40E+05 | 4.95E−04 | 2.06E−09 |
| L5A2 | M06 | 2.50E−07 | 8.54E+05 | 3.64E−03 | 4.26E−09 |
| L5A3 | KeF2b | 2.50E−07 | 1.08E+05 | 1.67E−04 | 1.55E−09 |
| | RB1.C12 | | | | |
| L5A4 | KeF2b | 2.50E−07 | 1.76E+05 | 5.83E−04 | 3.32E−09 |
| | RE1.B4b | | | | |

M03 and M06 are commercial antibodies. KeF1.RH1.H6 is immobilized at a concentration of 25 μg/ml. KeF1 protein is used at a concentration of 500 nM.

(2) M03 Immobilized, protein binding followed by sample (KeF2b.Rb1.C12, KeF2b.RE1.B4b and KeF1.RH1.H6)(FIG. 13)

| Param- | | Concen- | | | |
|---|---|---|---|---|---|
| eter | Sample | tration | ka | kd | KD |
| Unit | Name | M | 1/Ms | 1/s | M |
| L1A3 | KeF2b | 5.00E−07 | 1.99E+05 | 4.47E−05 | 2.25E−10 |
| | RB1.C12 | | | | |
| L1A4 | KeF2b | 5.00E−07 | 2.85E+05 | 1.27E−05 | 4.46E−11 |
| | RE1.B4b | | | | |
| L1A5 | KeF1 RH1.H6 | 5.00E−07 | 1.08E+04 | 4.25E−05 | 3.92E−09 |

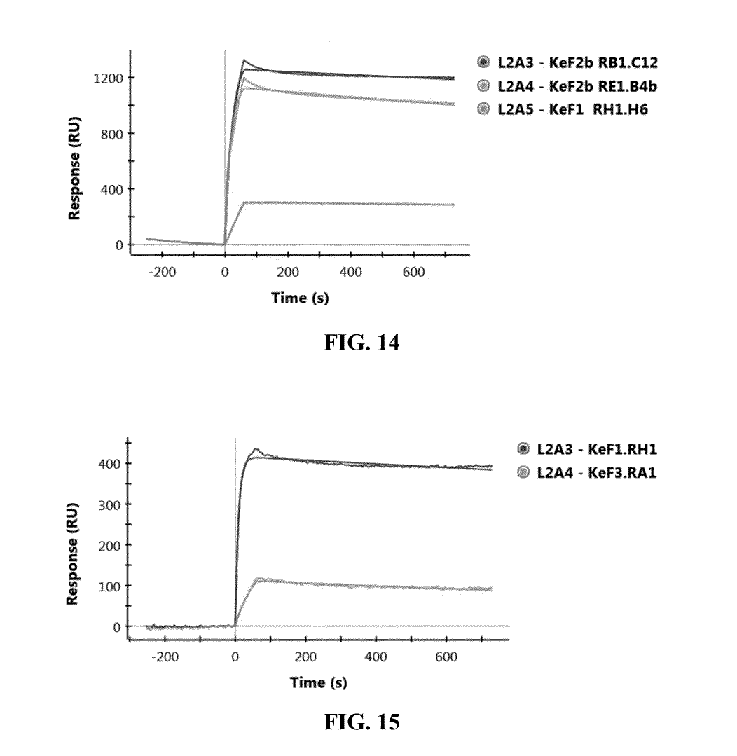

(3) M06 Immobilized, protein binding followed by sample (KeF2b.Rb1.C12, KeF2b.RE1.B4b and KeF1.RH1.H6)(FIG. 14)

| Param- | | Concen- | | | |
|---|---|---|---|---|---|
| eter | Sample | tration | ka | kd | KD |
| Unit | Name | M | 1/Ms | 1/s | M |
| L2A3 | KeF2b | 5.00E−07 | 9.05E+04 | 8.71E−05 | 9.63E−10 |
| | RB1.C12 | | | | |
| L2A4 | KeF2b | 5.00E−07 | 1.06E+05 | 1.74E−04 | 1.65E−09 |
| | RE1.B4b | | | | |
| L2A5 | KeF1 RH1.H6 | 5.00E−07 | 8.93E+03 | 8.56E−05 | 9.59E−09 |

(4) KeF2.RA1 immobilised, protein binding followed by sample KeF1.RH1 and KeF1.RA1 (preliminary data before sub cloning)(FIG. 15 )

| Param- | | Concen- | | | |
|---|---|---|---|---|---|
| eter | Sample | tration | Ka | kd | KD |
| Unit | Name | M | 1/Ms | 1/s | M |
| L2A3 | KeF1.RH1 | 5.00E−07 | 2.21E+05 | 1.12E−04 | 5.10E−10 |
| L2A4 | KeF3.RA1 | 5.00E−07 | 3.83E+04 | 3.52E−04 | 9.21E−09 |

1. A method of measuring fortilin in a blood sample comprising contacting a blood sample with an antibody that specifically binds fortilin forming an antibody/fortilin complex; contacting the antibody/fortilin complex with a detection reagent; and measuring the amount of detection reagent bound to the antibody/fortilin complex.

2. The method of claim 1, wherein the antibody that specifically binds fortilin is immobilized on a support.

3. The method of claim 2, wherein the support is a microtiter plate.

4. The method of claim 1, wherein the amount of detection reagent is determined by a chemical or enzymatic reaction.

5. A method for determining the level of in vivo apoptosis in a subject comprising measuring fortilin levels in a blood sample from the subject and determining whether the fortilin levels exceed a predetermined threshold representing levels of fortilin in a subject not having a disease, condition, or undergoing a therapy, wherein elevated fortilin levels are indicative of increased apoptosis associated with the disease, condition, or therapy.

6. The method of claim 5, wherein the cancer therapy is chemotherapy or radiation therapy.

7. The method of claim 5, wherein the disease or condition is cachexia, skeletal muscle atrophy associated with aging (sarcopenia), skeletal muscle atrophy associated with starvation, skeletal muscle atrophy associated with denervation, skeletal muscle atrophy associated with disuse, and skeletal muscle atrophy associated with inflammation.

8. The method of claim 5, wherein the subject is a human.
